(19) United States
(12) Patent Application Publication
(10) Pub. No.: US 2024/0124931 A1
(43) Pub. Date: Apr. 18, 2024

(54) MATERIAL AND METHOD FOR DIAGNOSIS OF TRAUMATIC BRAIN INJURY

(71) Applicants: Renato Rozental, Rio de Janeiro (BR); Pedro Henrique Freitas, Rio de Janeiro (BR)

(72) Inventors: Renato Rozental, Rio de Janeiro (BR); Pedro Henrique Freitas, Rio de Janeiro (BR)

(21) Appl. No.: 18/139,803

(22) Filed: Apr. 26, 2023

Related U.S. Application Data
(60) Provisional application No. 63/416,502, filed on Oct. 15, 2022.

(51) Int. Cl. C12Q 1/6876

(52) U.S. Cl. CPC C12Q 1/6876; C12Q 2600/158; C12Q 2600/178

(57) ABSTRACT

Materials and methods for the diagnosis of traumatic brain injury using miRNA biomarkers are disclosed. Amplification nucleotide chains amplify a detectable signal indicating the expression and/or upregulation of the biomarkers. Detection of the amplified signal is accomplished with capture nucleotide chains in a stem-loop conformation. The loop sequence of the capture nucleotide chains binds with the biomarkers and/or an indicator nucleotide chain released in the presence of one or more of the biomarkers. Binding is detected with indicators on one or more of indicator nucleotide chains released during amplification, the capture nucleotide chains, and signal nucleotide chains capable of complementary base-pair binding to the capture nucleotide chains. Incorporating the foregoing into a lateral flow assay permits point of care diagnosis.

### CROSS REFERENCE TO RELATED APPLICATION

This application claims the benefit of U.S. Provisional Application No. 63/416,502, filed on Oct. 15, 2022, the disclosure of which is hereby incorporated by reference in its entirety.

### FIELD OF THE INVENTION

Disclosed are nucleotide chains enabling detectable amplification of potent miRNA biomarkers, nucleotide chains for selective detection therefore, and a lateral flow assay for detecting such amplification in order to diagnose traumatic brain injuries.

### BACKGROUND

In the United States, it has been reported that traumatic brain injury results in millions of visits to hospitals and health care clinics each year. It is estimated, however, that traumatic brain injury affects over 60 million people annually. Accordingly, many are going untreated, due to the difficulty of properly diagnosing traumatic brain injury.

Most traumatic brain injuries, approximately 75%, fall into the category of concussion or mild traumatic brain injury. Despite the misnomer, mild traumatic brain injury can induce an intracranial event, such as subdural hematoma, epidural hematoma, subarachnoid hemorrhage, intraparenchymal hemorrhage, and/or brain edema. Symptoms of bleeding, however, may not be outwardly visible and/or present at the time of initial treatment.

The most efficacious time to address such serious symptoms is the “golden hour”, i.e., the first hour after receipt of the injury. Proper diagnosis facilitating such rapid treatment is hindered by the lack of outwardly visible symptoms. Additionally, though, CT scans and the detection of slow oscillations (or slow waves) via EEG or MEG can provide adequate diagnosis, such tests require specialized training to implement and interpret, and have other limitations, making them unsuitable for rapid and/or point of care diagnosis.

### SUMMARY OF THE INVENTION

Addressing the foregoing technical problems can be accomplished by utilizing potent miRNA biomarkers to diagnose concussion and/or other traumatic brain injury. Detection of the potent miRNA biomarkers in a patient is preceded by utilizing amplification nucleotide chains to amplify at room temperature a detectable signal indicating the expression and/or upregulation of the potent miRNA biomarkers. Selective and sensitive detection of the amplified signal provided by the amplification nucleotide chains is accomplished with capture nucleotide chains in a stem-loop conformation. Incorporating the capture nucleotide chains into a lateral flow assay permits accurate and rapid point of care diagnosis of concussion and/or other traumatic brain injuries. Capture nucleotide chains may comprise a loop sequence capable of complementary base-pair binding with the potent miRNA biomarkers and/or an indicator nucleotide chain released from the amplification nucleotide chains in the presence of one or more of the potent miRNA biomarkers. Binding of the potent miRNA biomarkers and/or the indicator nucleotide chains to the capture nucleotide chains may be detected with the aid of indicators on one or more of the indicator nucleotide chains released during amplification, the capture nucleotides, and signal nucleotide chains capable of complementary base-pair binding to the capture nucleotide chain after one or more of the following binds to the loop sequence of the capture nucleotide chain: an indicator nucleotide chain released during amplification, a nucleotide chain released during amplification that mimics one of the potent miRNA biomarkers, and one of the potent miRNA biomarkers.

At least a portion of the nucleotide chains captured by the capture nucleotide chains are released from amplification nucleotide chains during signal amplification triggered by the presence of one or more of the potent miRNA biomarkers. The capture nucleotide chains thus permit detection of signal amplification provided by the amplification nucleotide chains. Signal amplification by the amplification nucleotide chains occurs when one or more of the potent miRNA biomarkers is present. Thus, the amplification nucleotide chains and a lateral flow assay incorporating the capture nucleotide chains enable detection of the potent miRNA biomarkers within a sample retrieved from a patient.

The potent miRNA biomarkers comprise one or more of MIMAT0005878-hsa-miR-1287-5p, MIMAT0018079-hsa-miR-1273e, MIMAT0030415-hsa-miR-1273h-5p, MIMAT0027647-hsa-miR-6873-3p. These potent miRNA biomarkers can be used as an alternative for diagnosis via brain slow waves, an EEG abnormality preceding clinical symptoms, expansion of the primary lesion, and development of subsequent secondary lesions. As such, a lateral flow assay utilizing the capture nucleotide chains to detect signal amplification of the potent miRNA biomarkers via the amplification nucleotide chains enables a first responder with no specific medical skills to test for, identify, and within minutes after the occurrence of insult, detect a traumatic brain injury, such as concussion. When the lateral flow assay indicates expression and/or upregulation of one or more of the potent miRNA biomarkers, appropriate treatment for traumatic brain injury may be administered, such as, but not limited to, administration of acetaminophen to alleviate pain and/or reduce bleeding, administration of mannitol, hypertonic saline, and/or other therapeutics to reduce cerebral swelling, sedation, and/or removal from physical activity. As the potent miRNA biomarkers can cross the blood brain barrier, the lateral flow assay may be used with a variety of fluids, such as, but not limited to, saliva, blood, serum, urine, sweat, tears and amniotic fluid. More importantly, the lateral flow assay may be administered immediately following insult as to permit detection of traumatic brain injury within the “golden hour” for intervention and treatment.

These and other aspects and features of non-limiting embodiments of toehold nucleotides, capture nucleotides, and/or lateral flow assay relying thereon will become apparent to those skilled in the art upon review of the following description of specific non-limiting embodiments in conjunction with the accompanying drawings.

### BRIEF DESCRIPTION OF THE DRAWINGS

The drawings show various conceptual aspects of the invention. It should be understood, however, that the invention is not so limited to precise arrangements and instrumentalities shown in the drawings; wherein:

FIG. 1 depicts one method of collecting and preparing a sample;

FIG. 2 shows conceptually a substrate for the amplification of the potent miRNA biomarkers;

FIG. 3 shows conceptually a lower energy state adopted by the amplification substrate shown in FIG. 2 during amplification of a detectable signal;

FIG. 4 shows conceptually the displacement of an indicator nucleotide chain and a potent miRNA biomarker during amplification of a detectable signal;

FIG. 5 shows conceptually a substrate for secondary amplification of the signal provided by the substrate shown in FIG. 2;

FIG. 6 shows conceptually the displacement of a nucleotide chain mimicking at least one of the potent miRNA biomarkers and a different indicator nucleotide chain during secondary amplification of a detectable signal;

FIG. 7 shows conceptually a lateral flow assay for the point of care detection of traumatic brain injury;

FIG. 8 shows conceptually binding of a labeled indicator nucleotide chain to a capture nucleotide chain;

FIG. 9 shows conceptually binding of a signal nucleotide chain to a capture nucleotide chain to detect the capture of an unlabeled nucleotide chain;

FIG. 10 shows conceptually detectable binding of an unlabeled nucleotide chain to a label capture nucleotide chain;

FIG. 11 shows conceptually a PCB that may be used to detect and quantify binding to the capture nucleotide chains; and

FIG. 12 shows the sequence of the potent miRNA biomarkers.

The drawings are not necessarily to scale and are diagrammatic representations. Details that are not necessary for an understanding of the subject matter explained with the aid of the drawings and/or that render other details difficult to perceive may have been omitted.

### DETAILED DESCRIPTION

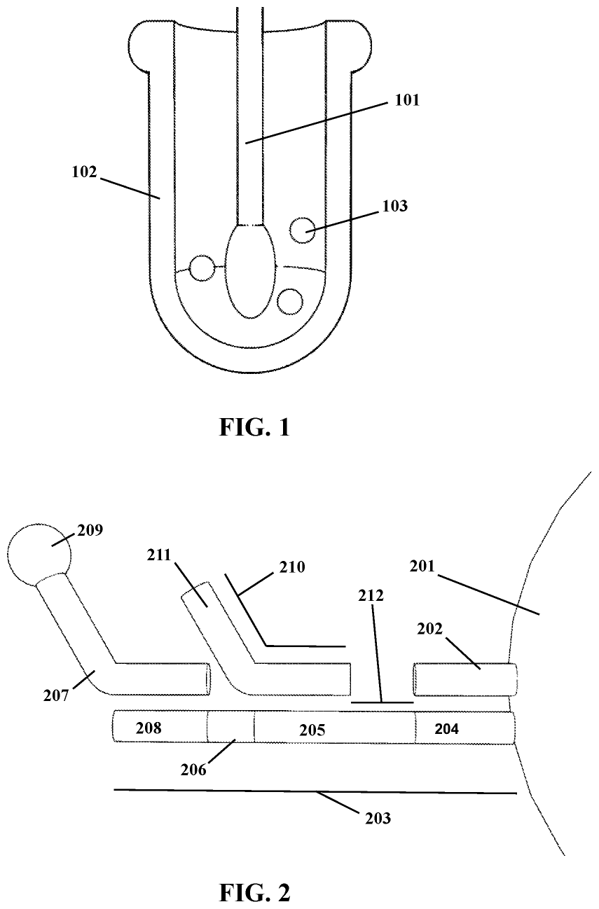

The potent miRNA biomarkers noted above enable point-of-care detection of concussion and/or other traumatic brain injuries using lateral flow assays. As shown in FIG. 1, point-of-care assays begin with collection and preparation of a sample. Collecting a sample from a patient may be as simple as collecting saliva from the patient's mouth with a swab 101. Once the sample has been collected, swab 101 may be placed in vial 102 containing a solution sufficient for removing miRNA from swab 101 so as to enable subsequent amplification and detection. The solution may be agitated as necessary. Besides saliva, other fluids may be collected from the patient as a sample, such as, but not limited to, blood, serum, saliva, urine, sweat, tears, and amniotic fluid. Fluorescence detection of miRNA can also be performed without amplification.

Selective amplification of miRNA biomarkers may occur simultaneously with treatment of the sample by including within vial 102 a substrate for the amplification of miRNA biomarkers carried on beads 103. As shown conceptually in FIG. 2, the substrate may comprise a surface 201 having a plurality of attached single-stranded nucleotide chains 202 attached to the surface at either their 5′ or 3′ end. For the sake of simplicity, FIG. 2 only shows one attached single-stranded nucleotide chain 202 of the plurality. When beads 103 comprise glass, or another material having hydroxyl groups, the attached single-stranded nucleotides may be attached via a silane-polyethylene glycol-N-hydroxysuccinimide. Briefly, the silane reacts with a hydroxyl of the surface, as to replace a methanol of the silane with the hydroxyl of the surface. A primary amine group on the to-be attached single-stranded nucleotide chain then reacts with the PEG to replace the hydroxysuccinimide. Other surfaces and methods of the attachment may be utilized, so long as they provide attached single-stranded nucleotide chains projecting from the surface.

One of a plurality of single-stranded amplification nucleotide chains 203 is attached to one of the plurality of attached single-stranded nucleotide chains 202 via complementary base pairing of an end anchor sequence 204 of the single-stranded amplification nucleotide chain 203. Thus, the end anchor sequence 204 of the single-stranded amplification nucleotide chain 203 forms a double-strand with one of the plurality of attached single-stranded nucleotide chains 202.

Adjacent to anchor sequence 204, each of the plurality of single-stranded amplification nucleotide chains 203 have one of a plurality of target sequences 205. Target sequences 205 permit one of the potent miRNAs biomarkers from the sample to form a double-strand with one of the plurality of single-stranded amplification nucleotide chains 203. As such, the plurality of target sequences 205 comprises: a first subset having a sequence complementary to that of MIMAT0005878-hsa-miR-1287-5p; a second subset having a sequence complementary to that of MIMAT0018079-hsa-miR-1273e; a third subset having a sequence complementary to that of MIMAT0030415-hsa-miR-1273h-5p; and a fourth subset having a sequence complementary to that of MIMAT0027647-hsa-miR-6873-3p.

Adjacent to target sequence 205, the single-stranded amplification nucleotide chains 203 have a toehold sequence 206. During amplification, toehold sequence 206 permits complementary base pairing with an indicator release nucleotide chain 302 as to form a double-strand releasing one of the plurality single-stranded indicator nucleotide chains 207. The to-be released indicator nucleotide chains 207 have a sequence complementary to an indicator binding sequence 208 of the plurality of single-stranded amplification nucleotide chains 203 as to permit each of the indicator nucleotide chains 207 to form a double-strand with the indicator binding sequence 208 of one of the plurality of amplification nucleotide chains 203. Indicator nucleotide chains 207 may have an attached indicator 209 permitting visualization or other detection of the respective indicator nucleotide chain 207. Alternatively, peptide-based miRNA detection methodology can be used.

In addition to amplification nucleotide chains 203 and attached single-stranded nucleotide chains 202, the amplification substrate shown in FIG. 2 also comprises plurality of single-stranded blocking nucleotide chains 210. Each of the blocking nucleotide chains 210 comprises a sequence complementary to the toehold sequence 206 and a sequence complementary to a portion of the target sequence 205 of one of the plurality of amplification nucleotide chains 203. Having adjacent sequences complementary to adjacent sequences of the amplification nucleotide chains 203 permits each of the plurality of blocking nucleotide chains 203 to form a double-strand with at least one of the amplification nucleotide chains 203 comprising the toehold sequence 206 and the target sequence 205. As noted above, the target sequence 205 of each amplification nucleotide chain 203 is complementary to either MIMAT0005878-hsa-miR-1287-5p, MIMAT0018079-hsa-miR-1273e, MIMAT0030415-hsa-miR-1273h-5p, and MIMAT0027647-hsa-miR-6873-3p. As such, each amplification nucleotide chain 203 contains one of four possible target sequences. As the miRNA biomarkers are unique at their ends, the plurality of blocking nucleotide chains 210 comprises: a first subset having sequence corresponding to a terminal portion of MIMAT0005878-hsa-miR-1287-5p; a second subset having a sequence corresponding to a terminal portion of MIMAT0018079-hsa-miR-1273e; a third subset having a sequence corresponding to a terminal portion of MIMAT0030415-hsa-miR-1273h-5p; and a fourth subset having a sequence corresponding to a terminal portion of MIMAT0027647-hsa-miR-6873-3p.

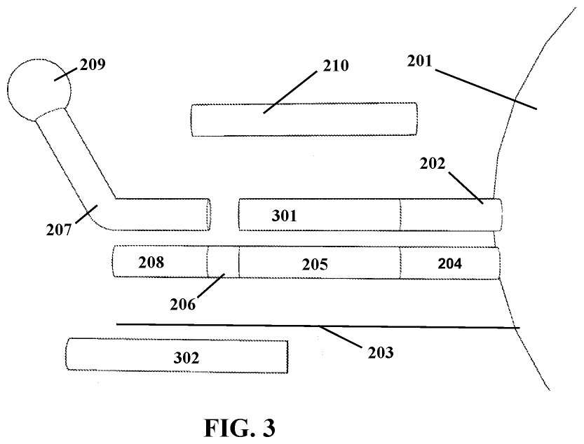

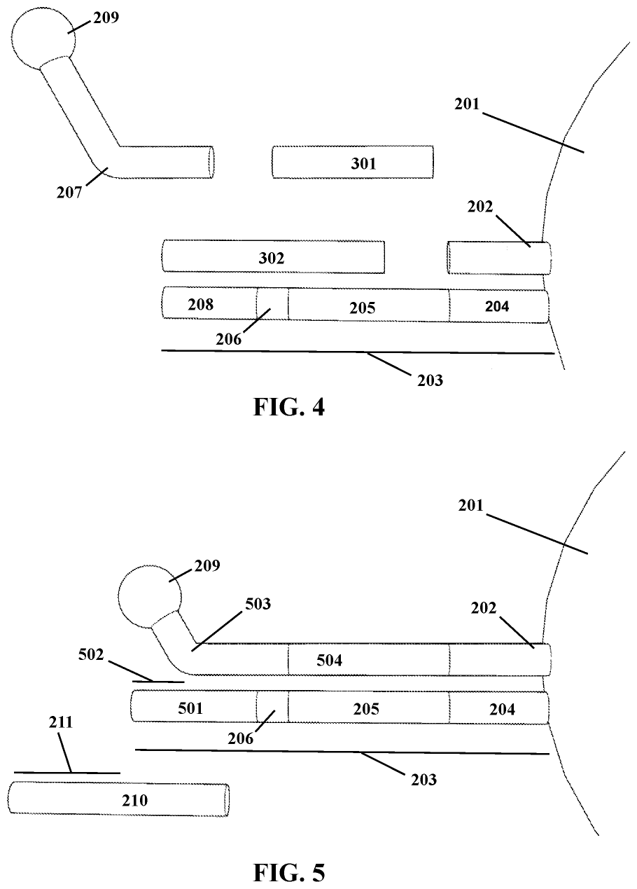

Selective amplification of the miRNA biomarkers is shown conceptually in FIGS. 3 and 4. As shown in FIG. 3, one of the miRNA biomarkers 301 forms a double-strand with its corresponding target sequence 205 of one of the plurality of amplification nucleotide chains 203 via complementary base pair binding. The sequences of the potent miRNA biomarkers 301 are shown in FIG. 12. Forming the double-strand between the miRNA biomarker 301 and target sequence 205 causes displacement of blocking nucleotide chain 210. The reduction in free energy disfavors the reverse reactions, i.e., the return of blocking nucleotide chain 210 and displacement of miRNA biomarker 301. As shown in FIG. 2, only a portion of blocking nucleotide chain 210 forms a double-strand with amplification nucleotide chain 203. A terminal portion 211 of each blocking nucleotide chain 210 remains unbound. Likewise, the binding of blocking nucleotide chain 210 to target sequence 205 of each amplification nucleotide chain 203 is incomplete, as the opposite terminal sequence of blocking nucleotide chain 210 is only complementary to a portion of target sequence 205. Therefore, when a blocking nucleotide chain 210 is bound to a amplification nucleotide chain 203 a single-stranded portion 212 of target sequence 205 is present. The single-stranded terminal portion 211 of blocking nucleotide chain 210 and the single-stranded portion 212 of target sequence 205 increases the free energy of the resulting complex shown in FIG. 2.

In the presence of miRNA biomarker 301 the complex can obtain the lower free energy state shown in FIG. 3. When present, the miRNA biomarker 301 binds to the single-stranded portion 212 of target sequence 205. The binding continues displacing blocking nucleotide chain 210 from target sequence 205. Once displaced, blocking nucleotide chain 210 can adopt the more relaxed confirmation shown in FIG. 3 by eliminating the kink preceding the single-stranded terminal portion 211. As such, the displacement of blocking nucleotide chain 210 from nucleotide chain 203 by miRNA biomarker 301 enables the system to obtain the lower free energy state shown in FIG. 3. However, the confirmation shown in FIG. 3 has the miRNA biomarker 301 sequestered to the complex via complementary base pair binding with amplification nucleotide chain 203. Furthermore, indicator nucleotide chain 207 is also attached to amplification nucleotide chain 203 via complementary base pair binding. The complex shown in FIG. 3, consequently, provides neither amplification nor indication of presence of miRNA biomarker 301. As such, it is necessary to displace the indicator nucleotide chain 207 and miRNA biomarker 301 with indicator release nucleotide chain 302 comprising a first sequence complementary to at least a portion of the target sequence 205 of one of the plurality of amplification nucleotide chains 203; a second sequence, adjacent the first sequence, complementary to the toehold sequence 206 of the one of plurality of amplification nucleotide chains 203; and a third sequence, adjacent the second sequence, complementary to the indicator binding sequence 208 of the one of the plurality of amplification nucleotide chains 203. As shown in FIG. 3, when miRNA biomarker 301 displaces blocking nucleotide chain 210 toehold sequence 206 of amplification nucleotide chain 203 becomes exposed. Indicator release nucleotide chain 302 may then attach to amplification nucleotide chain 203 by complementary base paring initiated at toehold sequence 206.

The complete complementary base paring binding of an indicator release nucleotide chain 302 to an amplification nucleotide chain 203 provides the complex shown FIG. 4. As shown in FIG. 4, the binding of indicator release nucleotide chain 302 causes the displacement of indicator nucleotide chain 207 and miRNA biomarker 301. To promote the release, and inhibit reattachment, the double-strand nucleotide formed by amplification nucleotide chain 203 and indicator release nucleotide chain 302 should have a lower free energy than that of the complex shown in FIG. 3. The release of indicator nucleotide chain 207 permits detection of the presence of miRNA biomarker 301. The release of miRNA biomarker 301 permits amplification of the signal. Once released, miRNA biomarker 301 is free to attach another amplification nucleotide chain 203 in the state shown in FIG. 2. This will result in the cycle shown in FIGS. 2-4 repeating, thereby causing the release of another indicator nucleotide chain 207. With each new cycle, the signal produced by the miRNA biomarker 301 is amplified.

Further amplification of the signal may be provided by including within the plurality amplification nucleotide chains 203 a subset of having a different indicator binding sequence 501 comprising a single-stranded overhang sequence 502, as shown conceptually in FIG. 5. As the amplification nucleotide chain 203 is of a subset having a different indicator binding sequence 501, it consequently has a different indicator nucleotide chain 503 attached via complementary base pair binding. As can be seen from FIG. 5, the different indicator nucleotide chain 503 has a sequence complementary to the toehold sequence of 206 of amplification nucleotide chain 203 and, adjacent thereto, a sequence complementary to a portion of different indicator binding sequence 501.

In addition to different indicator nucleotide chain 503, amplification nucleotide chain 203 also a mimics nucleotide chain 504 attached via complementary base pair binding with target sequence 205. As target sequence 205 of each amplification nucleotide chain 203 is complementary to either MIMAT0005878-hsa-miR-1287-5p, MIMAT0018079-hsa-miR-1273e, MIMAT0030415-hsa-miR-1273h-5p, and MIMAT0027647-hsa-miR-6873-3p, mimic nucleotide chain 504 will have a nucleotide sequence equivalent to one of MIMAT0005878-hsa-miR-1287-5p, MIMAT0018079-hsa-miR-1273e, MIMAT0030415-hsa-miR-1273h-5p, and MIMAT0027647-hsa-miR-6873-3p.

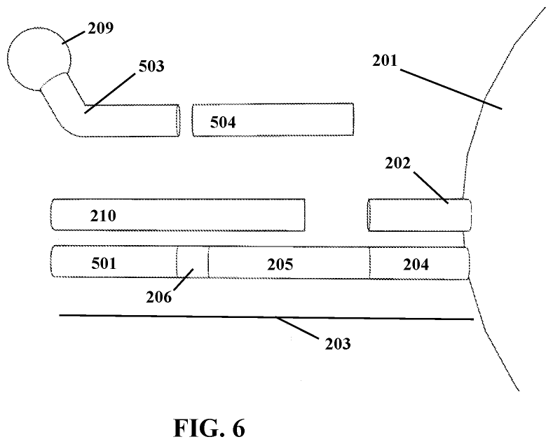

The overhang sequence 502 of the different indicator binding sequence 501 permits amplification of the signal by the blocking nucleotide chain 210 displaced from the complex shown in FIG. 2 by miRNA biomarker 301. The overhang sequence 502 is complementary to the terminal portion 211 of blocking nucleotide chain 210, thereby providing a toehold for the complementary base pair binding of blocking nucleotide chain 210 to amplification nucleotide chain 203. As shown conceptually in FIG. 6, the complementary base pair binding of blocking nucleotide chain 210 to amplification nucleotide chain 203 displaces different indicator nucleotide chain 503 and mimic nucleotide chain 504. As blocking nucleotide chain 210 is made available to form the complex shown in FIG. 6 upon the binding of miRNA biomarker 301 to the complex shown in FIG. 2, the signal indicating the presence of miRNA biomarker 301 is amplified. Additional amplification comes from the release of mimic nucleotide chain 504. Having nucleotide sequence equivalent to miRNA biomarker 301, once released mimic nucleotide chain 504 can bind via complementary base pair binding to the complex shown in FIG. 2, thereby inducing the release of another blocking nucleotide chain 210 and another indicator nucleotide chain 207 to further amplify the signal. As to inhibit miRNA biomarker 301 and/or mimic nucleotide chain 504 from displacing blocking nucleotide chain 210 from different indicator binding sequence 501, the binding of blocking nucleotide 210 to different indicator sequence 501, toehold sequence 206, and the terminal portion of target sequence 205 should more thermodynamically favored than the binding of either mimic nucleotide chain 504 or miRNA biomarker 301 to target sequence 205.

As noted above, the state shown in FIG. 4 is more thermodynamically favored than the state shown in FIG. 2. Likewise, the state shown in FIG. 6 if more thermodynamically favored than the state shown in FIG. 5. Accordingly, generation and amplification of the signal provided by the presence of miRNA biomarkers in the sample collected from the patient is driven by a change in free energy. Detectable amplification of the miRNA biomarkers, therefore, may be performed at room temperature and/or with mild heating. Such makes the amplification substrate suitable for point-of-care use to detect and/or diagnose concussion and/or other traumatic brain injury.

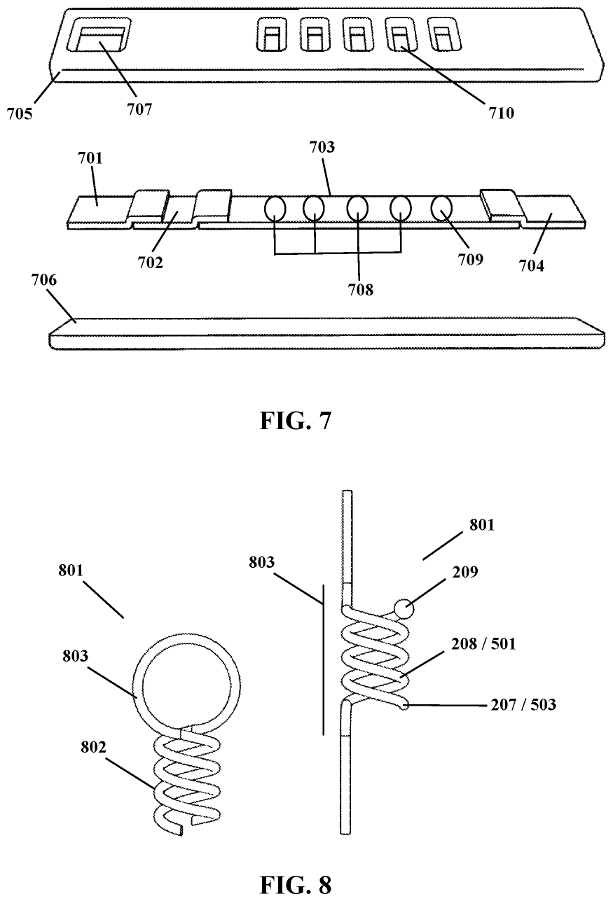

When present during the preparation of the sample in vial 102, the amplification substrate on beads 103 produces within vial 102 a solution containing miRNA biomarkers, indicator nucleotide chains, and/or mimic nucleotide chains, should any of the miRNA biomarkers be present in the sample. Point-of-care detection of all or a portion of these nucleotide chains may be achieved utilizing a lateral flow assay, such as that shown conceptually in FIG. 7. The lateral flow assay comprises a sample pad 701 of an adsorbent material, such as, but not limited to, cellulose or woven glass fibers, for receiving a fluid sample. The fluid from vial 102 may be used as the sample by placing all or portion of it on sample pad 701. Via capillary action, the fluid is drawn from sample pad 701 to conjugate release pad 702, which is positioned to receive the fluid from sample pad 701. Should the beads 103 not be present when the sample is extracted from swab 101 within vial 102, the conjugate release pad may comprise a plurality of attached single-stranded nucleotide chains 202 adhered to the conjugate release pad at either their 5′ or 3′ end, a plurality of single-stranded amplification nucleotide chains 203 attached thereto, and the plurality of single-stranded indicator nucleotide chains 207. Inclusion of amplification nucleotide chains 203 within conjugate release pad 702 may permit the signal amplification detailed above with respect to FIGS. 2-4 and/or FIGS. 5-6 to occur as the fluid is drawn through conjugate release pad 702. After being drawn through conjugate release pad 702, fluid enters membrane 703, which is positioned to receive fluid from the conjugate release pad. Membrane 703 may be formed from any adsorptive material, such as, but not limited to, nitrocellulose. The fluid is then drawn across membrane 703 by an adsorbent pad 704, which is positioned to receive the fluid from membrane 703. Adsorbent pad 704 may comprise cellulose, woven glass fibers, and/or any other material sufficiently adsorbent to draw fluid from sample pad 701. For ease of use, sample pad 701, conjugate release pad 702, and adsorbent pad 704 and membrane 703 may be placed within a housing comprising an upper shell 705 and a lower shell 706. When so housed, a fluid sample may be placed on sample pad 701 utilizing a sample through hole 707 within upper shell 705.

As the fluid is drawn across membrane 703, it passes through a capture region on membrane 703 having one or more test regions 708. Each test region 708 within the capture region comprises a plurality of single-stranded capture nucleotide chains, as shown conceptually in FIGS. 8-10. FIG. 8 conceptually shows an unlabeled capture nucleotide chain 801 attached to membrane 703 at either its 5′ or 3′ end. Initially, each of the capture nucleotide chains 801 is in a stem-loop conformation comprising stem 802 having a double-strand consisting of the 5′ end and 3′ end of the capture nucleotide 801 and a loop sequence 803 comprising the indicator binding sequence of one of the plurality of amplification nucleotide chains 203. Should amplification nucleotide chains 203 comprise a subset having different indicator binding sequence 501, then the plurality of capture nucleotide chains 801 should comprise a subset having different indicator binding sequence 501, as well as subset having indicator binding sequence 208. The stem-loop conformation of capture nucleotide chain 801 increases specificity with respect to indicator nucleotide chain 207 and/or different indicator nucleotide chain 503. As shown in FIG. 8, binding of either indicator nucleotide chain 207, 503 to its complementary sequence in loop sequence 803 disassociates the double-strand of stem 802. The complementary base pair binding of either indicator nucleotide chain 207, 503 to the loop sequence 803, thus, has to be more favorable than the complementary base pair binding of the capture nucleotide chain's 5′ and 3′ ends. Mismatched base pairs will make the binding of either indicator nucleotide chain 207, 503 less favorable, and thus less likely. The reduced probability of less favorable mismatched binding increases the selectivity of capture nucleotide chain 801 for the indicator nucleotide chains 207, 503. The indicator nucleotide chains 207, 503 shown in FIG. 8 comprise an indicator 209 permitting the complementary binding of either indicator nucleotide chain 207, 503 to capture nucleotide chain 801 to be observed through an indicator window 710 positioned within shell 705 above the corresponding test region 708. The indicator may be any molecule the accumulation thereof is capable of producing an observable change within a test region 708. For instance, the use of latex particles may provide an observable color change as indicator nucleotide chains 207, 503 bind to capture nucleotide chains 801, and thus accumulate in a test region 708. Observable fluorescent changes may be provided using fluorescent indicators 209.

As noted above, the release of either indicator nucleotide chain 207, 503 from an amplification nucleotide chain 203 is dependent on the presence of miRNA biomarker 301 in the sample, which may be one of MIMAT0005878-hsa-miR-1287-5p, MIMAT0018079-hsa-miR-1273e, MIMAT0030415-hsa-miR-1273h-5p, and MIMAT0027647-hsa-miR-6873-3p. Consequently, the subsequent complementary base pair binding of either indicator nucleotide chain 207, 503 to capture nucleotide chain 801 is dependent on the presence of one or more of MIMAT0005878-hsa-miR-1287-5p, MIMAT0018079-hsa-miR-1273e, MIMAT0030415-hsa-miR-1273h-5p, and MIMAT0027647-hsa-miR-6873-3p within the sample. As such, any observable change within a test region 708 indicates the presence of at least one of miRNA biomarkers 301 within the sample. Discerning which of the miRNA biomarkers are present may be accomplished by dividing the plurality of amplification nucleotide chains 203 into subsets such that each subset has a different indicator binding sequence 208, or collection thereof if further amplification via the mechanism shown in FIGS. 5 and 6 are to be used, unique to the particular target sequence 205 of the subset. When the plurality of amplification nucleotide chains 203 is so divided, the presence of each of the miRNA biomarkers 301 can be detected when the plurality of capture nucleotide chains 801 is likewise divided into subsets having the different indicator binding sequences of the plurality of amplification nucleotide chains 203. A simple visual detection of the different miRNA biomarkers 301 within the sample may be provided by sequestering the different subsets of capture nucleotide chains 801 to different test regions 708. Accordingly, membrane 703 may have first test region 708 having a first subset of the plurality of capture nucleotide chains 801 with loop sequences comprising one or both of indicator binding sequences 208, 501 of the first subset of amplification nucleotide chains 203, a second test region 708 having a second subset of the plurality of capture nucleotide chains 801 with loop sequence 803 comprising one or both of the indicator binding sequences 208, 501 of the second subset of amplification nucleotide chains 203, a third test region 708 having a third subset of the plurality of capture nucleotide chains 801 with loop sequences comprising one or both of the indicator binding sequences 208, 501 of the third subset of amplification nucleotide chains 203, and a fourth test region 708 having a fourth subset of the plurality of capture nucleotide chains 801 with loop sequences comprising one of both or the indicator binding sequences 208, 501 of the fourth subset of amplification nucleotide chains 203. With such an arrangement, a change observed in all four of the test regions 708 would indicate the presence of all four miRNA biomarkers 301 within the sample.

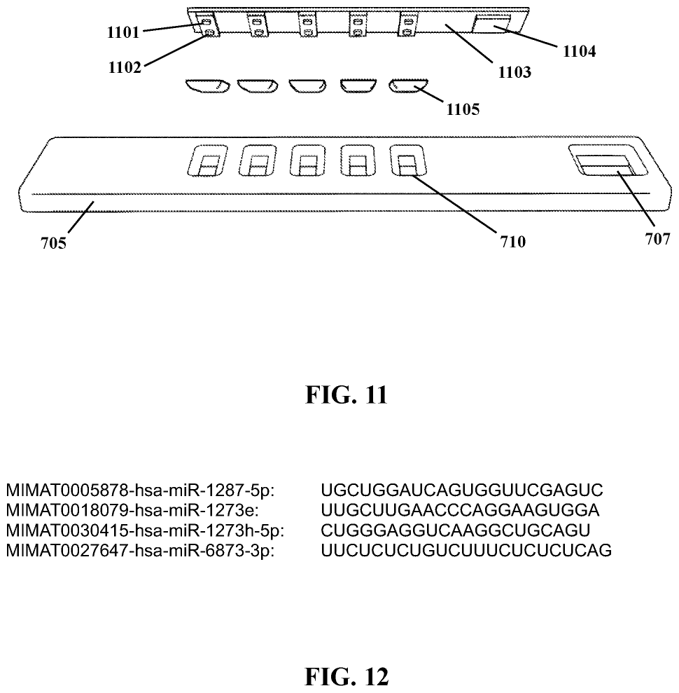

Discerning which of the miRNA biomarkers 301 are present within the sample may be accomplished by providing unique indicators 209 to one or more of the subsets of indicator nucleotide chains 207, 503 unique to the particular target sequence 205 of the subset of amplification nucleotide chains 203. For instance, each subset may be provided with an indicator 209 providing a unique color change to a test region 708, an indicator 209 emitting a unique wavelength of light, and/or an indicator 209 providing fluorescent when stimulated with a unique wavelength of light. When fluorescent indicators are utilized, it may be beneficial to use PCB 1103 having one or more light emitting diodes 1101 and one or more photoreceptors 1102 mounted thereon to be positioned over a test region 708, as shown in FIG. 11. To better spread the light emitted from diode 1101 over a test region 708, a lens 1105 capable of diffusing the emitted light may be placed within the corresponding indicator window 710. Lens 1105 may also facilitate the transmission of emitted fluorescence to photoreceptor 1102. Control of the diodes 1101 and/or quantification of the emitted fluorescence detected by photoreceptors 1102 may be provided by a microprocessor 1104. The number of test regions 708 necessary to distinguish the presence of the different miRNA biomarkers 301 can be reduced by emitting more than one wavelength of light over a given test region 708 and detecting multiple emitted fluorescence signals from a given test region 708. Such may be accomplished by positioning over a test region 708 one or more light emitting diodes 1101 collectively capable of emitting more than one wavelength of light and one or more photoreceptors 1102 collectively capable of detecting the emitted fluorescence. Accordingly, if each subset of indicators 209 emits fluorescence when excited with one of four wavelengths of light, a single test region may be utilized if one or more diodes 1101 collectively capable of emitting each wavelength of light is placed over the test region 708. Of course, the physical sequestering into test regions 708 may be combined with fluorescence such that a combination of fluorescence and location indicates the presence of a distinct miRNA biomarker 301 within the sample. Alternatively, miRNA detection can also be performed by a chromatographic method.

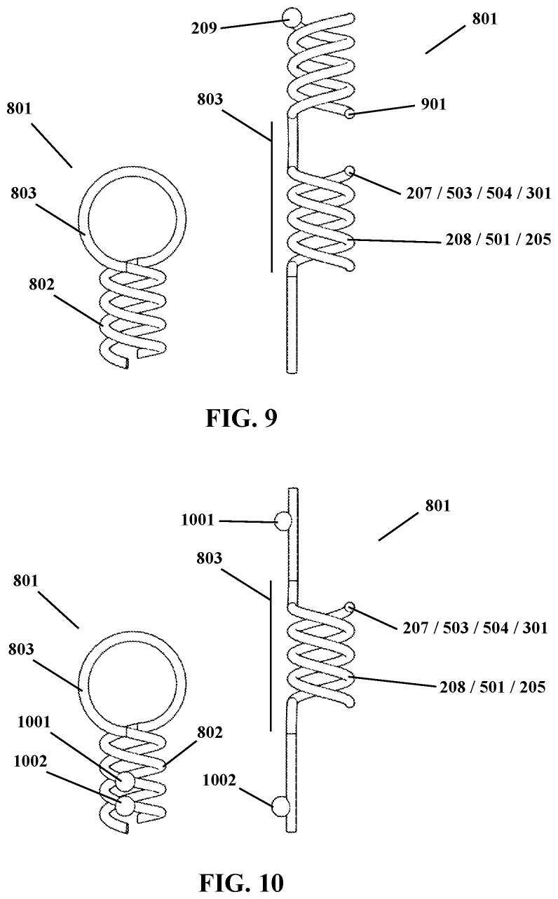

Indicator nucleotide chains 207, 503 need not be labeled with an indicator 209 when paired with a single-stranded signal nucleotide chain 901, as shown conceptually in FIG. 9. The indicator nucleotide chains 207, 503 lack an indicator. Rather, indicator 209 is attached to single-stranded signal nucleotide chain 901. As before, the complementary base pair binding of indicator nucleotide chains 207, 503, with indicator binding sequences 208, 501 within loop sequence 803 cause capture nucleotide chain 801 to adopt an open conformation. Adopting the open conformation causes the double-strand of stem 802 to disassociate, exposing the 5′ and 3′ ends of capture nucleotide 801. As shown conceptually in FIG. 9, a single-stranded signal nucleotide chain 901, comprising a sequence complementary to either the 5′ or 3′ end of the capture nucleotide forming the double-stranded stem 802, may attach to its complementary exposed end via complementary base pair binding. Signal nucleotide chain 901 further comprise indicator 209, allowing its binding to be detected. As indicator nucleotide chains 207, 503 are released from their respective amplification nucleotide chains by the presence of one of the miRNA biomarkers 301 in the sample, capture nucleotide chains 801 will adopt the open confirmation when one or more of miRNA biomarkers 301 is present within the sample. As adoption of the open conformation by capture nucleotide chain 801 is necessary for binding of signal nucleotide chain 901, the binding of signal nucleotide chain 901 to capture nucleotide chain 801 is dependent upon the presence of at least one of the miRNA biomarkers 301 within the sample. The accumulation of a detectable signal from indicator 209 attached to signal nucleotide chain 901 within a test region of the 208 of membrane 703, accordingly, indicates the presence of one or more of the miRNA biomarkers 301 within the sample. A plurality of single-stranded signal nucleotide chains 901 may be placed within conjugate release pad 702 as to be displaced by and carried with a fluid traversing conjugate release pad 702 and membrane 703.

Unlabeled indicator nucleotide chains 207, 503 are not the only molecules that may be detected by using signal nucleotide chains 901. It is also possible to detect the presence of one or more of the miRNA biomarkers 301 directly and/or indirectly via mimic nucleotide chains 504. As shown conceptually in FIG. 4, the complementary base-pair binding of release nucleotide chain 302 to nucleotide chain 203 causes the release of miRNA biomarker 301 along with indicator nucleotide chain 207. The fluid traversing membrane 703 will thus contain at least a portion of the miRNA biomarkers 301 present in the sample. Should the signal amplification shown in FIGS. 5 and 6 be utilized, mimic nucleotide chains 504, having the sequence of one or more of the miRNA biomarkers 301, will also be present in the fluid traversing membrane 703. The presence of mimic nucleotide chains 504 and miRNA biomarkers 301 may be detected by including within the capture region of the membrane 703 a second plurality of single-stranded capture nucleotide chains 801 comprising a 5′ end and a 3′end, where each of the capture nucleotide chains is in a stem-loop conformation comprising a stem having a double-stranded stem 802 consisting of the 5′ end and the 3′end of the capture nucleotide chain, and a loop sequence 803 comprising a target sequence 205 complementary to at least one of MIMAT0005878-hsa-miR-1287-5p, MIMAT0018079-hsa-miR-1273e, MIMAT0030415-hsa-miR-1273h-5p, MIMAT0027647-hsa-miR-6873-3p. Capture nucleotide chains 801 within any test region 708 of the membrane 703 may therefore comprise a loop sequence 803 having one of an indicator binding sequence 208, a different indicator binding sequence 501, and a target sequence 205, as to bind one of an indicator nucleotide chain 207, a different indicator nucleotide chain 503, a miRNA biomarker 301, and mimic nucleotide chain 504. Distinguishing which of the nucleotide chains is bound may be accomplished by sequestering capture nucleotide chains 801 into different test regions 708 based on their loop sequences 803 and/or by utilizing a plurality of single-stranded signal nucleotide chains having distinguishable indicators 209.

Capture nucleotide chains 801 may be configured to emit fluorescence when in the open conformation, as shown conceptually in FIG. 10, such that one or more of the plurality of capture nucleotide chains 801 may comprise a fluorescent probe 1001 and a quencher 1002 positioned on the capture nucleotide chain 801 as to quench fluorescent probe 1001 when the capture nucleotide chain 801 is in stem-loop conformation. When the capture nucleotide chain 801 adopts the upon conformation upon complementary base pair binding within loop sequence 803, fluorescent probe 1001 is sufficiently separated from quencher 1002 as to be positioned to emit fluorescence. As capture nucleotide chain 801 adopts the open conformation upon binding of a nucleotide chain to loop sequence 803, emission of fluorescence by probe 1001 would indicate the presence of specific nucleotide chain within the fluid traversing membrane 703. Of course, the nucleotide chains within the fluid traversing the membrane 703 would be dependent upon the miRNA biomarkers 301 present in the sample. As to enable direct and/or indirect detection of one or more of the miRNA biomarkers 301 within the sample, capture nucleotide chains 801 having probe 1001 and quencher 1002 may therefore comprise a loop sequence 803 having one of an indicator binding sequence 208, a different indicator binding sequence 501, and a target sequence 205, as to bind one of an indicator nucleotide chain 207, a different indicator nucleotide chain 503, a miRNA biomarker 301, and mimic nucleotide chain 504. Distinguishing which of the nucleotides is present may be accomplished by sequestering fluorescent capture nucleotide chains 801 into different test regions 708 based on their loop sequences 803 and/or by utilizing a plurality of fluorescent capture nucleotide chains 801 having distinguishable probes 1001.

To ensure proper movement of the sample fluid from sample pad 701 to absorbent pad 704, membrane 703 may also include a control region 709. Within the control region 709 may be a capture nucleotide chain having loop sequences capable of binding a control nucleotide sequence within the sample fluid and/or within the conjugate release pad 702. To determine if proper signal amplification is taking place via the mechanisms shown in FIGS. 2-4 and/or FIGS. 5-6, a plurality of amplification nucleotide chains having target sequences for commonly expressed miRNA may be included. The subsequent detection of control indicator nucleotide chains, control mimic nucleotide chains, and/or control miRNA biomarkers by control capture nucleotides in control region 709 would indicate proper functioning of the lateral flow assay and/or signal amplification.

The foregoing detailed nucleotide chains, substrates, and/or latera flow assays permit a method of treating traumatic brain injury in a patient in need thereof. The method begins by first identifying a traumatic brain injury in a patient by detecting in a fluid obtained from the patient the expression and/or upregulation of one or more of miRNA biomarkers selected from the group consisting of MIMAT0005878-hsa-miR-1287-5p, MIMAT0018079-hsa-miR-1273e, MIMAT0030415-hsa-miR-1273h-5p, and MIMAT0027647-hsa-miR-6873-3p. When such an expression and/or upregulation is detected, thereby indicating the presence of traumatic brain injury, the patient may be treated for the traumatic brain injury by administering one or more of acetaminophen; a therapeutic to reduce cerebral swelling, sedation, and removal from physical activity. In some instances, the presence of traumatic brain injury may be detected by detected by the expression and/or upregulation of each of the miRNA biomarkers MIMAT0005878-hsa-miR-1287-5p, MIMAT0018079-hsa-miR-1273e, MIMAT0030415-hsa-miR-1273h-5p, and MIMAT0027647-hsa-miR-6873-3p. When necessary, the therapeutic to reduce cerebral swelling may be mannitol and/or hypertonic saline.

The foregoing has been a detailed description of the illustrative embodiments. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. A substrate for the amplification of biomarkers indicative of traumatic brain injury, the substrate comprising:
a surface comprising a plurality of attached single-stranded nucleotide chains, each of the single-stranded nucleotide chains comprising a 5′ end and a 3′ end opposite the 5′ end, and each of the attached single-stranded nucleotide chains attached to the surface at either their 5′ or 3′ end;
a plurality of single-stranded amplification nucleotide chains, each of the amplification nucleotide chains comprising:
an end anchor sequence complementary to at least one of the plurality of attached single-stranded nucleotide chains attached to the surface, the end anchor sequence forming a double-strand with one of the plurality of attached single-stranded nucleotide chains attached to the surface;
one target sequence of a plurality of target sequences, the one target sequence adjacent to the end anchor sequence, the plurality of target sequences comprising:
a first subset having a sequence complementary to that of MIMAT0005878-hsa-miR-1287-5p;
a second subset having a sequence complementary to that of MIMAT0018079-hsa-miR-1273e;
a third subset having a sequence complementary to that of MIMAT0030415-hsa-miR-1273h-5p; and
a fourth subset having a sequence complementary to that of MIMAT0027647-hsa-miR-6873-3p;
a toehold sequence adjacent the one target sequence; and
an indicator binding sequence adjacent the toehold sequence; and
a plurality of single-stranded indicator nucleotide chains comprising:
a sequence complementary to the indicator binding sequence of at least a portion of the plurality of amplification nucleotide chains,
wherein each of the plurality of indicator nucleotide chains forms a double-strand with the indicator binding sequence of one of the plurality amplification nucleotide chains.

2. The substrate of claim 1 further comprising an indicator attached to each of the plurality of single-stranded indicator nucleotide chains.

3. The substrate of claim 1 further comprising:
a plurality of single-stranded blocking nucleotide chains, each of the single-stranded blocking nucleotide chains comprising:
a sequence complementary to the toehold sequence of the amplification nucleotide chains; and
a sequence complementary to at least a portion of the target sequence of at least one of the plurality of amplification nucleotide chains,
wherein each of the plurality of blocking nucleotide chains forms a double-strand with the toehold sequence and a portion of the target sequence of one of the plurality of amplification nucleotide chains.

4. The substrate of claim 1, wherein the plurality of single-stranded amplification nucleotide chains further comprises:
a first subset of amplification nucleotide chains; and
a second subset of amplification nucleotide chains having a different indicator binding sequence than the first subset of amplification nucleotide chains.

5. The substrate of claim 4, wherein at least a portion of the different indicator binding sequence of the second subset of amplification nucleotide chains comprises a single-stranded overhang sequence.

6. The substrate of claim 5 further comprising:
a plurality of single-stranded mimic nucleotide chains comprising a sequence complementary to the target sequence of at least one of the plurality of amplification nucleotide chains,
wherein each of the plurality of mimic nucleotides forms a double-strand with the target sequence of one amplification nucleotide chains of the second subset of amplification nucleotide chains.

7. The substrate of claim 5 further comprising:
a plurality of single-stranded blocking nucleotide chains, each of the single-stranded blocking nucleotide chains comprising:
a sequence complementary to the different indicator binding sequence of the second subset of amplification nucleotide chains;
a sequence complementary to the toehold sequence of the plurality of amplification nucleotide chains; and
a sequence complementary to at least a portion of the target sequence of one of the plurality of amplification nucleotide chains,
wherein, each of the plurality of blocking nucleotide chains forms a double-strand with the toehold binding sequence and a portion of the target sequence of one of the plurality amplification nucleotide chains of the first subset amplification nucleotide chains.

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)


SEQUENCE LISTING (4 sequences)

SEQ ID NO: 1 - RNA, 22 nt, Homo sapiens
  source 1..22: mol_type = other RNA
tgctggatcagtggttcgagtc

SEQ ID NO: 2 - RNA, 22 nt, Homo sapiens
  source 1..22: mol_type = other RNA
ttgcttgaacccaggaagtgga

SEQ ID NO: 3 - RNA, 21 nt, Homo sapiens
  source 1..21: mol_type = other RNA
ctgggaggtcaaggctgcagt

SEQ ID NO: 4 - RNA, 23 nt, Homo sapiens
  source 1..23: mol_type = other RNA
ttctctctgtctttctctctcag